(19) United States
(12) Patent Application Publication
(10) Pub. No.: US 2006/0078618 A1
(43) Pub. Date: Apr. 13, 2006

(54) Lipid particles and suspensions and uses thereof

(76) Inventors: Panayiotis P Constantinides, Gurnee, IL (US); Reena T Patil, Palatine, IL (US); Likan Liang, Boyds, MD (US)

Correspondence Address: KATTEN MUCHIN ROSENMAN LLP, 525 WEST MONROE STREET, CHICAGO, IL, 60661-3693, US

(21) Appl. No.: 10/498,572

(22) PCT Filed: Dec. 11, 2002
(86) PCT No.: PCT/US02/39661

§ 371 Date: May 27, 2005

Related U.S. Application Data
(60) Provisional application No. 60/339,811, filed on Dec. 11, 2001.

(51) Int. Cl. A61K 9/14

(52) U.S. Cl. 424/489

(57) ABSTRACT

The present invention relates to formulations and methods for the mucosal and parenteral administration of lipid particles an suspensions. The formulations of this invention are stable lipid particles useful for oral delivery of water-insoluble therapeutic agents, vaccines and diagnostics. The compositions of this invention promote the mucosal absorption of biologically active molecules across mucosal epithelial barriers. Stabilization of lipid particles is achieved by coating the hydrophobic central core with a polymer shell. The polymer shell can include bioadhesive agents, ligands, and absorption promoting agents. This invention relates to oral drug delivery systems for hydrophobic drugs, and in particular is concerned with improving the bioavailability of hydrophobic drugs from such systems. Using this system, anticancer drugs such as taxanes are orally effective.

### BACKGROUND

1. Field of the Invention

The present invention relates to delivery systems for the mucosal and parenteral administration of biologically active molecules, including, but not limited to, therapeutic agents, vaccines, allergens, antigens and diagnostic agents. In particular, the present invention relates to lipid suspension and lipid particle compositions comprising lipids, polymerized lipids and derivatives thereof, fatty acid esters and derivatives thereof, additional water miscible solvents and surfactants, and optionally one or more biologically active agents, and methods of administering biologically active molecules to an animal utilizing said compositions. The compositions of this invention further comprise polymeric ingredients to promote stability in the gastrointestinal tract and in contact with mucosal fluids. The compositions of the invention promote the absorption of biologically active molecules across mucosal epithelial barriers and are especially suitable for promoting the absorption of poorly water-soluble drugs, such as paclitaxel and other taxanes. The compositions of the invention can be used therapeutically, diagnostically or cosmetically.

2. Background of the Invention

Many drugs are limited in their development by the parenteral route of administration. Thus one of the great challenges in the improvement of the therapeutic potential of poorly water-soluble drugs is the development of systems that will provide optimal solubility and oral or mucosal bioavailability. Drugs, compounds, biologically active agents and the like with little or no solubility in water are also referred to as lipophilic or hydrophobic and these terms are indistinguishable within the scope of the present invention.

The most convenient way to administer drugs into the body is by oral administration. However, many drugs, in particular water-insoluble drugs and macromolecular compounds such as proteins and peptides, are poorly absorbed or unstable during passage through the gastrointestinal (GI) tract. The administration of these poorly absorbed or unstable drugs is generally performed through parenteral injection. Many of the more recently developed anticancer agents, though effective, can only be administered via intravenous injection because of low solubility in water. A large proportion of the macromolecular drugs developed by recombinant DNA methods can be delivered only by injection of the molecules, either subcutaneously or through intravenous administration.

Lipid systems have been widely exploited for development of drug delivery vehicles and systems. Most of the lipid-based particulate systems that have been developed for delivery of poorly water-soluble, or lipophilic drugs, are micellar, emulsion or suspension type formulations. For many hydrophobic drugs, there remains the need to find a carrier system that will enhance the bioavailability of such drugs in the GI tract.

One approach to making suitable formulations of water-insoluble drugs is to solubilize a hydrophobic therapeutic agent in an oil and disperse this oil phase in an aqueous phase. Depending on whether an oil is a solid or liquid at the ambient temperature, the oil-in-water emulsion can be characterized as a solid lipid particulate. The dispersion may be stabilized by emulsifying agents and provided in emulsion form. In a water milieu, drugs dissolved in the oil phase or the solid lipid core can be dispersed by mechanical force to create microdroplets or microspheres that are stable in storage as a pharmaceutical preparation. The formation of a stable oil-in-water emulsion or lipid spheres depends on the use of surfactants that form the interface between the strictly hydrophobic oil and water. Depending on the nature of the oil and co-surfactant, either large droplets or particles are formed. Further control over size of droplets or particles can be obtained by high pressure homogenization or similar shear forces. Lipid particles are typically formed at higher ambient temperatures to melt the hydrophobic components.

Oil-in-water (o/w) emulsions are also commonly formed from oil(s), surfactant(s), and an aqueous phase. Typically, oils are used in drug delivery systems to solubilize lipophilic drugs and to make them more effective and less toxic. Oils used in typical emulsions are any of a number of oils such as mineral, vegetable, animal, essential and synthetic oils, or mixtures thereof. In many cases oils rich in triglycerides, such as safflower oil, cottonseed oil, olive oil or soybean oil are used. Such emulsions contain the hydrophobic therapeutic agent solubilized in an oil phase that is dispersed in an aqueous environment with the aid of a surfactant or a combination of surfactants. Therefore, one approach to making suitable formulations of hydrophobic drugs is to solubilize a hydrophobic drug in an oil and to disperse this oil phase in an aqueous phase.

For many hydrophobic therapeutic agents, solubility is too low in aqueous solution to offer formulations that can deliver therapeutically effective doses. Hydrophobic therapeutic agents, while poorly soluble in aqueous solution, could be sufficiently lipophilic that therapeutically effective concentrations can be prepared in triglyceride-based solvents. The colloidal oil particles sizes are relatively large, ranging from several hundred nanometers to several microns in diameter, in a broad particle size distribution. Although triglyceride-based pharmaceutical compositions are useful in solubilizing and delivering some hydrophobic therapeutic agents, such compositions are subject to a number of significant limitations and disadvantages. Under most conditions, emulsions are thermodynamically unstable, the droplet spontaneously agglomerating, eventually leading to complete phase separation. The tendency to agglomerate and phase separate presents problems of storage and handling, and increases the likelihood that pharmaceutical emulsions initially properly prepared will be in a less optimal, less effective, and poorly-characterized state upon ultimate administration to a patient.

In other cases, therapeutic compounds, although hydrophobic, are insufficiently soluble in tri-glycerides and cannot be formulated solely in triglyceride oils. Surfactants are also required to form solid lipid suspension. And the same forces that operate in liquid oil phase also cause the precipitation of hydrophobic drugs at the interface of lipids with water upon short or long term storage and destabilize lipid particle suspension systems.

Although many lipid-based systems are used to promote absorption of drugs with poor water solubility, lipid-based systems still have several drawbacks in the development of orally or mucosally delivered drugs. Most lipid systems comprise triglycerides which are readily digested in the milieu of the digestive system. Upon digestion of the triglycerides, the encapsulated hydrophobic therapeutic agent may precipitate and may not be absorbed. If absorption of the hydrophobic therapeutic agent occurs, the digestion of the triglycerides also causes rapid rather than sustained absorption.

Lipid-based delivery systems such as emulsion and microemulsions systems, or lipid particulate systems are based on the use of polar lipids and related amphiphilic surfactant molecules to control the interaction of hydrophobic molecules with water. In many cases, delivery systems for hydrophobic drugs have also required the inclusion of organic solvents that are water miscible in order to increase the molecular interactions between drugs and lipid or surfactant components.

Lipid-based delivery systems may additionally incorporate absorption enhancers, such as the salicylates, bile salts and other surfactants. Absorption enhancers may function to increase the permeation of peptide, protein, and lipophilic molecules across epithelial barriers because of their interaction with the GI mucosa and concomitant opening of the tight junctions. A wide variety of amphiphilic molecules are known to behave as absorption enhancers. In addition, bile salts and salicylates, medium chain fatty acid salts and esters, and medium chain monoglycerides and di-glycerides are known to have mucosal absorption enhancing activity. Absorption enhancement with these molecules is attributed to the presence of medium chain C₆-C₁₂ fatty acyl chains (6-12 carbon atoms in length), particularly those esterified with C₈-C₁₀ fatty acids (8-10 carbon atoms in length).

Lipids and surfactants are differentiable from short and long chain hydrocarbons in that they are amphiphilic molecules, having both hydrophilic and hydrophobic moieties. Surfactants are conveniently classified on an empirical scale known as the hydrophile-lipophile balance (HLB) which runs from about 1 to about 45 for ionic surfactants and from about 1 to about 20 for non-ionic surfactants. HLB values closer to 1 represent surfactants with more lipophilic character, while HLB values that are greater than about 10 represent more hydrophilic surfactants.

Most familiar in the class of lipid vehicles are liposomes. Liposomes are traditionally formed from pure or mixed phospholipids or mixtures of phospholipds with cholesterol or fatty acids. The characteristic feature of liposomes is the formation of an interfacial bilayer membrane that separates an internal water compartment from the external water milieu. Drugs and other active materials can be entrapped within the internal aqueous space. Conventional liposomes have been used successfully to develop commercial pharmaceutical compositions that abrogate the toxicity of certain drugs such as amphotericin, when administered intravenously. A major problem encountered with the development of liposomes as drug delivery vehicles is their poor ability to withstand exposure to stomach acids, bile salts and phospholipases. For development of stable lipid drug delivery vehicles, polymerized liposome systems have been developed. Most of the work with polymerized lipid particles has been focused on polymerized liposomes, where a polymerizable phospholipid or fatty acid is incorporated in the lipid bilayer of the liposome which upon polymerization produced a crosslinked bilayer with increased rigidity and physical stability.

U.S. Pat. No. 5,160,740 (Hasegawa, E., et al.) discloses polymerization of a 3,5 polymerizable 2,4-diene phospholipid, cholesterol, and a polymerizable 2,4-diene fatty acid to form a polymerized macromolecular endoplasmic reticulum. Additionally, U.S. Pat. No. 5,762,904 (Okada, J., et al.) discloses the use of polymerized liposomes for the delivery of oral vaccines. Polymerized liposomes are formed using a bilayer forming phospholipid or mixtures of said phospholipids with non-phospholipid structures. The presence of the polymer phospholipid results in a membrane that resists dissolution by detergents and bile salts and is more acid resistant. A number of additional polymerizable phospholipids are described in Regan, in Liposomes: from Biophysics to Therapeutics (Ostro, ed., 1987), Marcel Dekker, N.Y.

Incorporating a targeting ligand on the surface of a liposome may increase the efficiency of absorption of drugs encapsulated in those liposomes. U.S. Pat. No. 6,004,534 (Langer, R. S. and Chen, H.) discloses modifications to the surface of polymerized liposomes in which plant lectins were conjugated. Such lectins recognize receptors on the surface of epithelial cells and promote greater adherence of the liposomes to M cells (Chen et al., 1996, Pharmaceutical Research 13:1378-1383).

Additional strategies to enhance the bioavailability of hydrophobic drugs include methods to increase surface area of drug crystals and the co-inclusion of P-glycoprotein (PGP) inhibitors in formulations in an effort to increase absorption. Many drugs are substrates for the PGP, which acts as an efflux pump. As disclosed in U.S. Pat. No. 6,245,8,05 (Broder, S., K. L. Duchin, and S. Selim/Baker Norton Pharmaceuticals, Inc.), cyclosporin A may be used to enhance the bioavailability of hydrophobic drugs by inhibiting PGP. Additional compounds that are known inhibitors of PGP can also be used to enhance the bioavailability of lipophilic drugs. These include other PGP inhibitors such as cyclosporine analogues, surfactants such as poloxamers, polysorbates, α-tocopherol polyethylene glycol esters, as well as therapeutic agents known to affect the activity of PGP such as verapamil and ketoconazole.

U.S. Pat. No. 6,207,178 (K. Westesen) discloses solid lipid particles and particles of bioactive agents and methods for the manufacture and use thereof. A suspension formulation is manufactured by an emulsifying process resulting in non-spherical particles (anisometric). These solid lipid particles provide a controlled release dosage form for poorly water soluble drugs primarily by IV but also by peroral, nasal, pulmonary, rectal, dermal, and buccal route of administration. Water insoluble drugs can be melted in the lipid phase prior to homogenization. The melted lipid phase is emulsified in the aqueous phase using high pressure homogenization or sonication. These particles can be freeze dried by removing the liquid using ultrafiltration.

WO 00/06120 (Jeong, S., et al.) discloses lipid emulsion a and solid lipid nanoparticle as a gene or drug carrier. The emulsion is an oil-in-water lipid emulsion which is composed of non-triglyceride oils and also solid lipid nanoparticles (SLN) composed of triglyceride or ethyl stearate, phospholipids and non-ionic surfactants used as gene transfection agents and drug delivery systems. The SLN are prepared without melting of the lipids, by mixing the aqueous and the fatty phases.

U.S. Pat. No. 5,904,932 (T. DeVringer) discloses a preparation containing a suspension of solid lipid particles made of lipid and emulsifier for topical application.

U.S. Pat. No. 5,726,164 (Weder, H. G. and P. van Hoogevest) discloses nanosuspensions for intravenous administration. Pharmaceutical composition containing the active which is insoluble (specifically N-benzoyl-staurosprin), a polyoxyethylene/polyoxypropylene block copolymer, purified lecithin, water soluble excipients glycerol and sorbitol along with ethanol and water as solvents for the IV administration of insoluble drugs.

U.S. Pat. No. 5,188,837 (A. J. Domb) discloses lipospheres for controlled delivery of substances. A microsuspension containing lipospheres which are solid, water insoluble particles that have a layer of a phospholipid embedded on their surface. The lipid core is a solid substance to be delivered that is dispersed in wax.

For conventional oral delivery systems, the drug is released into the GI tract within a short period of time, and plasma drug levels peak usually within a few hours after dosing. A controlled release oral dosage form is designed to maintain drug levels at constant effective concentrations. Generally, controlled delivery of lipophilic drugs requires techniques different than those employed with hydrophilic drugs. Lipophilic drugs must be solubilized in order to be released in a controlled fashion.

Citation or identification of any reference in Section 2, or any section of this application shall not be construed as an admission that such reference is available as prior art to the present invention.

### SUMMARY OF THE INVENTION

The present invention is directed to a particle composition that comprises a hydrophobic phase, a surfactant, and a polymeric stabilizer. The composition may optionally comprise a biologically active agent. The composition may also optionally comprise a solvent. The hydrophobic phase of the composition may comprise an oil or a mixture of oils. A preferred oil is a triglyceride. Preferably the hydrophobic phase comprises a triglyceride that is a liquid between 10° C. and 45° C., and more preferably is a hydrophobic phase comprising a triglyceride that is liquid between 10° C. and 70° C.

The surfactant of composition may be alkyl glycerolphosphoryl choline, a polyoxyethylene polymer, a block copolymer of polyoxyethylene and polyoxypropylene, or an ethoxylated glycerol ester. The surfactant may also comprise one or more glycerol fatty acid esters or hydrophilic derivatives thereof. Preferably the fatty acid esters have a length of about 6 to about 12 carbon atoms. In another embodiment, the surfactant may comprise a monoglyceride, diglyceride, or a hydrophilic derivative or analog thereof.

A preferred monoglyceride useful in the practice of the present invention, is a monoglyceride derivatized by a polyoxyethylene polymer of about 200 to about 10,000 daltons in molecular weight. Preferably the polyoxyethylene polymer is from about 200 to about 4,000 in molecular weight. A preferred diglyceride useful in the practice of the present invention is a diglyceride derivatized by a polyoxyethylene polymer. Fatty acid esters useful in the present invention include fatty acid esters derivatized with acetic acid, citric acid, lactic acid, succinic acid, tartaric acid, or mixtures thereof. A preferred fatty acid ester useful in the practice of the present invention is a caprylic acid or capric acid. Preferred surfactants have a HLB value of about 1 to about 45 and preferably from about 1 to about 20.

Solvents useful in the practice of the present invention include water, glycerol, sorbitol, mannitol, propylene glycol, ethylene glycol, polyethylene glycol, or mixtures thereof. In a preferred embodiment, the solvent of the present invention comprises monoterpene or a derivative thereof. Preferred monoterpenes includes perillyl alcohol, perilladehyde, perillic acid, perillilic acid methyl ester and d-limonene.

The particles of the present invention may be suspended in an aqueous phase comprising an additive. Preferred additives include a suspending agent, buffering agent, tonicity agent, an oxidizing agent, a reducing agent, an antimicrobial agent, a preservative, a stabilizing agent or mixtures thereof. In the compositions of the present invention, the hydrophobic phase is preferably present in an amount from about 0% to about 50% by weight and more preferably in amount from about 10% to about 25% by weight. Preferred biologically active molecules useful in the compositions of the present invention include prophylactics or therapeutic agents. They may also be a diagnostic agent. A preferred therapeutic agent is a taxane or an analog thereof. A more preferred biologically active molecule is paclitaxel.

In another embodiment of the present invention the biologically active molecule is a topoisomerase inhibitor selected from the group consisting of etoposide, camptothecin, topotecan, or a derivative thereof. In compositions of the present invention, the biologically active molecule is preferably soluble at least 0.1 mg/ml in the hydrophobic phase, and more preferably soluble at least 1.0 mg/ml in the hydrophobic phase. The polymeric stabilizers useful in the compositions of the invention include but are not limited to natural polymers, synthetic polymers, or mixtures thereof. The polymeric stabilizers include polymerizable fatty acids or polymerizable phospholipids. The more preferred polymeric stabilizers are ODP or OPS. Preferably the polymer may be formed by interfacial ionic polymerization with water or other initiators. However, the polymer may also be formed by condensation of cyanoacrylates, including but not limited to alkyl cyanoaciylates. A preferred alkyl cyanoacrylate is ethyl 2-cyanoacrylate. The synthetic polymer may also be a polylactide, a polyglycolide, a mixture of polylactide and polyglycolide, a polycaprolactone, a polyortho ester, polysebacic acid, polyfumaric acid, polyamides, polycarbonates, polyalkylenes, polyacrylamides, poly(hydroxy acids), polyanhydrides, polyortho esters, polyacrylate, polyvinyl alcohols, blends or copolymers thereof.

The particle composition of the pi-sent invention may also be in the form of a suspension. Preferably the size of the particles in the suspension range from about 10 nm to about 10,000 nm and are preferably m the range of 10 nm to 1,000 nm. The present invention is also directed to methods for delivering biologically active methods to an animal, methods for treating, preventing, or ameliorating one or more symptoms of a disease in an animal, and methods for diagnosing a disease or disorder in an animal using any of the foregoing compositions. The compositions of the present invention may be administered in the form of a capsule, soft elastic gelatin capsule, caplet, aerosol, spray, solution, suspension, emulsion, sachet, tablet, powder, or granules. The composition is preferably administered orally.

### DETAILED DESCRIPTION OF THE INVENTION

Many systems for oral delivery of hydrophobic drugs are oil-based wherein the hydrophobic drug being is dissolved in an oil. However, the administration of a drug in oil alone is not advantageous because of the poor miscibility of the oil with the aqueous environment of the gastrointestinal tract The present invention provides a stable lipid suspension. The lipid suspensions of the present invention may be given to an animal alone or in combination with water-insoluble molecules, e.g. lipophilic drugs, for treating, preventing or diagnosing disease states. The lipid suspension of this invention is effective in promoting the absorption of hydrophobic biologically active materials by mucosal tissues by protectively encapsulating one or more hydrophobic materials within a stable lipid particle comprised of one or more hydrophobic solvents, one or more surfactants, and one or more polymeric stabilizers. More than one drug or pharmaceutical agent and/or formulation at a time can be used according to the present invention to yield a desired pharmaceutical composition. Also, in accordance with the present invention is a method of treating disease, such as cancer, using a drug delivery system for increasing the bioavailability of one or more hydrophobic drugs.

The lipid particles of this invention are stabilized by a polymer or oligomer shell, which encases the hydrophobic core of the liquid particle. The polymer or oligomer shell is comprised of one or more polymeric stabilizers which have undergone polymerization or cross-linking. The porosity of the polymeric shell is controlled by the degree of polymerization or cross-linking of the polymeric stabilizers. The lipid particles and suspensions of the present invention provide improved bioavailability of hydrophobic compounds because hydrophobic drugs are contained in the central hydrophobic core of the lipid particle and are prevented from contacting aqueous solution, such as gastrointestinal or mucosal fluid. In addition, the encapsulated hydrophobic drugs are able to interact directly with the membrane barriers of absorptive cells. Bioavailability of hydophobic drugs can also be increased by conjugating absorption promoting agents to the polymeric compounds that form the shell of the lipid core. In addition to increase bioavailabilty, the polymeric shell provides increased stability of lipid suspensions in aqueous storage milieu and increased stability upon lyophilization. Absorption promoting compounds may be compounds that control the opening and closing of intercellular tight junctions or that interact with epithelial cell receptors. In addition, absorption promoting compounds may be compounds that are bioadhesive or mucoadhesive.

Stabilization of the lipid particles can be achieved by forming hydrophobic biologically active molecule-containing particles in the presence of monomeric polymerizable compounds and subsequent polymerization in situ. Polymerization in situ results in a polymer network surrounding the central drug-containing core of the lipid particle. Alternatively, stabilization of the hydrophobic core of a lipid particle can be achieved by the addition of hydrophobic polymers that interact with the hydrophobic moieties of the core-forming materials. The polymeric materials add physical rigidity to the system by interacting with acyl side chains of the hydrophobic phase materials to impede the dissolution of the hydrophobic phase by enzymes or detergents in mucosal fluids. When polymerized, lipid particles have greater stability in vitro upon contact with water or simulated or actual gastrointestinal fluid.

U.S. Pat. No. 6,187,335 (Brey, R. N., L. Liang) discloses polymerizable fatty acid compounds. These compounds are aliphatic fatty acids with polymerizable groups in the head group or in the aliphatic chain. Such fatty acids are further modifiable by extension of their hydrophilic head groups by ethylene glycol addition or addition of other hydrophilic groups. The structure of these fatty acids gives them unique functionality and particular utility when used in conjunction with lipid particles.

The fatty acids described by Brey and Liang are fully compatible with amphiphilic and lipid materials and when contacted with water form thermodynamically stable structures. When used with lipid particles formed with oils as the principal hydrophobic phase, such fatty acids form the outer shell with the polar head groups of the fatty acid oriented towards the water phase and the hydrophobic side chains contained in the hydrophobic compartment. Polymerization of the fatty acids results in crosslinked lipid in which the fatty acid polymer stabilizes the lipid particle.

The surfactant group of the fatty acids is disposed between the polymerizable group and the functional acid group, which can be optionally omitted. The length of the polymeric chain of the surfactant group can be chosen to be short, medium or long, in order to control the relative hydrophilicity/hydrophobicity of the chain. A long-chain surfactant group with significant hydrophilicity, for example, may provide hydrophilic groups that interact effectively with cell membranes, may provide in and of itself the surfactant activity, or may contain ligands that specifically bind with cellular receptors.

For the purpose of forming polymers in situ in the lipid particle or suspension, polymerization of the fatty acid polymerizable moiety may be carried out using three methods: (1) actions of chemical initiators, e.g., redox pairs; (2) physical excitation, including sensitized photoinitiation, e.g., broad band UV or UV 360 nm or UV 302 nm irradiation, gamma-ray irradiation, cyanine dye with an argon laser; and (3) combination of chemical initiators with physical excitation. Methods of initiating polymerization may result in harsh environment changes, such as large pH drops and inactivation of protein drugs. As a result, the most desirable method of polymerization initiation is where polymerization can be controlled and the activity of therapeutic materials is totally retained.

Precise control of polymerization level is sometimes difficult to achieve with the use of chemical initiators. In addition, additional steps are normally required to separate any unreacted initiators, especially when low level of polymerization is needed. Diene polymerizable functions may be polymerized by exposure to short-wave or mid-wave ultraviolet light. Ultraviolet light at 302 nm may be used to polymerize diene function and damage to proteins and peptides can be minimized. Phenylacetophenone initiators combined with UV 360 nm irradiation has been used extensively in the polymerization of alkeno functionalities, such as acrylated PEG hydrogel for biomedical and molecular imprinting applications, polymethacrylate polymers for biomaterials and tissue engineering, and styrene/acrylate/methacrylate nanoparticles for drug delivery. Long range UV wavelengths are usually outside of the absorption range of proteins and would not cause damage to proteins.

One embodiment of this invention is a lipid particle comprising one or more hydrophobic solvents, one or more surfactants, one or more biologically active hydrophobic molecules, and a polymeric shell, said polymeric shell comprised of one or more polymeric stabilizers. A preferred hydrophobic solvent is an oil. The lipid particle of the present invention is a spherical or non-spherical particle with a diameter preferably between about 1 nm to about 1000 nm, and more preferable with a diameter between 10 nm and 1000 nm, and most preferably with a diameter between 50 nm and 500 nm.

The stable lipid particle according to the present invention comprises one or more hydrophobic solvents, preferably an oil that is a liquid at room temperature or an oil that is a liquid at body temperature or above. A drug or pharmaceutical with poor solubility in water may be solubilized in the hydrophobic solvent and can subsequently be dispersed in an aqueous phase for administration to an animal.

In a preferred embodiment of the invention, the hydrophobic solvent is present in an amount of about 5% to about 50% of the total weight of the lipid particle. In a more preferred embodiment of the invention, the hydrophobic solvent is present in an amount of about 10% to about 40% of the total weight of the lipid particle. In the most preferred embodiment of the invention, the hydrophobic solvent is present in an amount of about 20% to about 30% of the total weight of the lipid particle.

The lipid particle according to the present invention comprises one or more surfactants. The surfactants of the current invention can include emulsifying agents with HLB values from 1-45, from 1-20, from 5-20, from 5-15. Typically, surfactants or emulsifiers within HLB in the range of 5-20 can be used. In the present invention, the preferred HLB range for the surfactant(s) is between approximately 5 and 15. Preferred surfactants utilized in accordance with the present invention include lecithn. Other preferred surfactants include ethoxylated derivatives of medium chain mono- and di-glycerides such as Labrasol (caprylic/capric (C₈/C₁₀) polyethylene glycol mono- and di-glycerides, Gattefosse Corporation). Any suitable surfactant may be employed alone or in combination with other surfactants. For example, egg yolk phospholipids such as lecithin or polyethylene oxide-polypropylene oxide block copolymers (Pluronics) such as Pluronic F68 (Poloxamer 188), having a molecular weight of about 8,000. Other suitable Pluronics, include Pluronic F87 (Poloxamer 237) with an average molecular weight of about 7,500 and Pluronic F127(Poloxamer 407) with an average molecular weight of about 12,000. Ethoxylated diacyl glycerol and dialkyl ether glycerol are useful surfactants. The lipid particles of this invention may contain alkylphosphoryl choline or alkylglycerophosphoryl choline and other lipid surfactants such as 1,2-dioctylglycero-3-phosphoryl choline, 1,2-ditetradecylglycero-3-phosphoryl choline, 1,2-dihexadecylglycero-3-phosphoryl choline, 1,2-dioctadecylglycero-3-phosphoryl choline, 1-hexadecyl-2tetradecylglycero-3-phosphoryl choline, 1-octadecyl-2-tetradecylglycero-3-phosphoryl choline, 1-tetradecyl-2-octadecylglycero-3-phosphoryl choline, 1-hexadecyl-2-octadecylglycero-3-phosphoryl choline, 1-2-dioctadecylglycero-3-phosphoryl choline, 1-octadecyl-2-hexadecylglycero-3-phosphoryl choline, 1-tetradecyl-2-hexadecylglycero-3-phosphoryl choline, 2,2-ditetradecyl-l-phosphoryl choline ethane, and ethoxylated derivatives of medium chain mono- and di-glycerides. Anionic surfactants with HLB values preferably greater than about 10, such as alkyl or aryl sulfates, sulfonates, carboxylates or phosphates, and cationic surfactants with HLB values preferably greater than about 10, such as mono-, di-, tri- and tetraalkyl or aryl ammonium salts may also be used in the practice of the present invention. Non-ionic surfactants such as polysorbate 80 (Tween 80) and polyalcohols such as polyvinyl alcohol may also be used. Zwitter-ionic surfactants that have a combination of the anionic or cationic groups, and whose hydrophobic part consists of any other polymer, such as polyisobutylene or polypropylene oxides, may also be used. Mixtures of these surfactants may also be used as may other surfactants well known in the art. The surfactant of this invention has HLB values preferably from 1-20, more preferably from 5-20, and even more preferably from 5-10 or from 10-15.

In a preferred embodiment of the invention, the surfactant is present in an amount of about 15% to about 90% of the total weight of the lipid particle. In a more preferred embodiment of the invention, the surfactant is present in an amount of about 10% to about 30% of the total weight of the lipid particle. In the most preferred embodiment of the invention, the surfactant is present in an amount of about 20% to about 30% of the total weight of the lipid particle.

The lipid particle according to the present invention also comprises a biologically active hydrophobic molecule. Such hydrophobic molecules include but are not limited to methotrexate, cis-platin and derivatives, vincristine, vinblastine, quinolone, ciprofloxacin, progesterone, teniposide, estradiol, doxorubicin, epirubicin, taxanes and topoisomerase inhibitors. Other hydrophobic molecules useful in the practice of the present invention include prostaglandins, amphotericin B, testosterone, beclomethasone and esters, vitamin E, cortisone, dexamethasone and esters, betamethasone valerete and other steroids, nifedipine, griseofulvin, cyclosporin, digoxin, itraconozole, carbamazepine, piroxicam, fluconazole, indomethacin, steroids, ibuprofen, diazepam, finasteride and diflunisal. Preferred topoisomerase inhibitors include etoposide, camptothecin, topotecan, or derivatives thereof. Combinations of more than one hydrophobic drug or pharmaceutical ingredient with poor solubility in water may be formulated according to the present invention to yield a desired pharmaceutical composition.

The lipid particle according to the present invention also comprises a polymeric shell. Such polymeric shell is a microstructure comprising one or more polymeric stabilizers which have undergone polymerization and/or cross-linking. The polymeric shell is formed by means including, but not limited to chemical initiation with oxidation reduction initiators, or high energy radiation such as gamma or ultraviolet irradiation, and combinations thereof In a preferred embodiment of this invention, the polymeric shell is formed by irradiation by ultraviolet light at either 302 nm or 350 nm in the presence of chemical initiators.

Polymeric stabilizers preferably form microstructures such as microparticles, microtubules, microspheres, matrices, and microcrystals that are compatible with the hydrophobic phase of the surfactant mixture may be used to stabilize the lipid particles and suspension compositions. Such nicrostructures encapsulate the lipid particle within their structure. A polymeric stabilizer may be a natural polymer, a synthetic polymer or a mixture thereof.

Polymeric stabilizers according to the invention include but are not limited to polylactide, poly-glycolide, a mixture of polylactide and polyglycolide, a hydrocarbon oligomer, a hydrocarbon polymer, a polycaprolactone, a polyortho ester, polysebacic acid, polyfumaric acid, a polyamide, a polycarbonate, a polyalkylene, a polyacrylamide, poly(hydroxy acid), a polyanhydride, a polyorthoester, blends and copolymers thereof. Other polymeric stabilizers useful in the practice of the invention include fatty acid or fatty acid derivatives or phospholipid or phospholipid derivatives that are polymerizable, 2,4 octadecadienoic acid [ODA],2,4 octadecadienoyl-polyethylene glycol (200-4000) [ODP],2,4 octadecadienoyl-PEG (2004,000)-succinic acid [OPS], Bis-(2,4-octadecadienoyl)-polyethylene glycol (200-10,000) [BODP] and analogs thereof. Appropriate analogs include analogs modified by single amino acids or polypeptide chains, imido groups, polyamines, polyimines, polysaccharides, polyacids, polymers or co-polymer of propylene glycol and ethylene glycol. Other polymerizable moieties may also be used, including, conjugated dienes of C6-C24, conjugated diynes of C6-C24, methacrylate modified or sulfhydryl-containing polar groups or hydrophobic tails of the fatty acids. The use of 2,4 conjugated dienes results in polymers that are linked to adjacent acyl groups in the side chains in the internal hydrophobic phase, where use of sulfhydryl containing polymerizable fatty acids results in head group polymerization at the interface of the aqueous and hydrophobic phases.

In addition, other polymerizable fatty acid derivatives can be used to stabilize the lipid particles. The polar head group, for example, may consist of amino acids, polypeptides, polysaccharides, polyols, polyacrylic acids, polyimines, choline, peptidoglycols, glycopeptides, or other hydrophilic polymers with multiple positive or negative charges. Further, compounds that are polymerizable fatty acid derivatives of glycerol or glyceryl phosphatidyl derivatives compatible with the hydrophobic lipid core may be used.

In a preferred embodiment of this invention, the polymeric stabilizer is DODPC (2,4 dioctacedadienoyl phosphatidyl choline). In another preferred embodiment of this invention, a second polymeric stabilizer is a polymerizable fatty acid with polyethylene glyccol polar head groups or a polymerizable phopholipid. In a more preferred embodiment of this invention, the polymeric stabilizer ODP (2,4 octadecadienoyl-polyethylene glycol −200-4000).

In another preferred embodiment of this invention, the polymeric shell comprises a polymerizable fatty acid monomer or derivative, or monomers which have undergone interfacial ionic polymerization with water, such condensation of cyanoacrylates, alkylcyanoacrylates (e.g. ethyl 2-cyanoacrylate).

In addition to the above constituents, the lipid particle of this invention may contain a secondary surfactant or “co-surfactant”. Such secondary surfactants include but are not limited to any of the surfactants described above, as well as Labrasol (Gattefosse Corporation), which is comprised of a mixture of capric caprylic (C₈-C₁₀) mono- and di-glycerides triglycerides. The secondary surfactant of this invention has HLB values preferably from 5-20, more preferably from 5-15, and even more preferably from 5-10 or from 10-15.

In a preferred embodiment of the invention, the secondary surfactant is present in an amount of about 16% to about 89% of the total weight of the lipid particle. In the most preferred embodiment of the invention, the secondary surfactant is present in an amount of about 10% to about 40% ofthe total weight of the lipid particle.

In addition to the above constituents, the lipid particle of this invention may contain a second hydrophobic solvent, which is miscible in the hydrophobic solvent described above. The second hydrophobic solvent may further solubilize the hydrophobic biological agent Such second hydrophobic solvents include but are not limited to polyethylene glycol, glycerol and related esters of fatty acids, polymerizable fatty acids, or polymerizable lipids, and monoterpene alcohols such as perillyl alcohol or lirnonene.

In addition to the above constituents, the lipid particle of this invention may contain a hydrophobic polymer. Hydrophobic polymers are insoluble in water and soluble in organic solvents. Such hydrophobic polymers include, but are not limited to polymers comprising polylactic acid, polyglycolic acid, polyorthoesters, polysebacic acid, polymethyl methacrylate, polyacrylates, polystyrenes, and polyfumarate. The aliphatic chains of hydrophobic polymers interact primarily with the hydrophobic side chain of oils and surfactants of the hydrophobic core of the lipid phase to form a loose network of polymer chains thereby stabilizing the hydrophobic core of a lipid particle.

In addition to the above constituents, the lipid particle of this invention may contain, monomeric compounds. Such monomeric compounds include but are not limited to members of the cyano-acrylate family, such as 2-cyanoacrylate (ECA). Monomeric compounds that undergo polymerization in contact with water may be used to create polymers at the interface of the aqueous phase and the hydrophobic phase. For example, a solution of 2-cyanoacrylate (ECA) dissolved in methylene chloride can be added to the hydrophobic phase of a preformed lipid core. Upon stirring, ECA contacts the aqueous phase and polymerization is initiated. The removal of solvent by evaporation results in polymerization of ECA into a polymer principally at the interface of the lipid particle and water interface.

In addition to the above constituents, the lipid particle of this invention may contain other lipidic compounds. Such lipidic compounds include, but are not limited to, low melting temperature waxes, including N.F. White Beeswax, Soy wax, Carnuba wax, Castor wax, Microwax, and other such waxes. Such waxes can be melted and mixed directly with polymeric stabilizers such as mono- or di-glyceride fatty acid esters.

The lipid particles and suspensions according to the present invention also comprise a hydrophobic therapeutic agent. Such hydrophobic therapeutic agents include but are not limited to methotrexate, cis-platin and derivatives, vincristine, vinblastine, quinolone, ciprofloxacin, progesterone, daunorubicin, teniposide, estradiol, doxorubicin, epirubicin, and taxanes. Other hydrophobic therapeutic agents useful in the practice of the present invention include prostaglandins, amphotericin B, testosterone, beclomethasone and esters, vitamin E, cortisone, dexamethasone and esters, betamethasone valerete and other steroids, nifedipine, griseofulvin, cyclosporin, digoxin, itraconozole, carbamazepine, piroxicam, fluconazole, indomethacin, steroids, ibuprofen, diazepam, finasteride and diflunisal. Other therapeutic agents may also be used including antibiotics (antiviral, antibacterial, antihelminthic, antiplasmodial, or antimycotic), analgesics and local anesthetics, antidepressants, antipsychotics, sedatives, hypnotics, hormones, cytokines, vaccine adjuvants and antigens, immunosuppressive agents, vasodilators, antiarrhythmics, calcium antagonists, cardiac glycosides, oligonucleotides, oligopeptides, anti-emetics, and migraine therapeutics.

The lipid particles and suspensions according to the present invention may also comprise hydrophilic therapeutic molecules that can be derivatized with a hydrophobic compound. Upon derivatization with a hydrophobic compound, the hydrophilic molecule may be solubilized within the hydrophobic phase of the lipid particle. Methods of derivatization include but are not limited to conjugation of fatty acids through ester linkages to the amino terminal amino acid of a peptide or to epsilon amino groups of lysines resulting in esterification of acyl chains to proteins and peptides. Further, oligosaccharides and polysaccharides may be derivatized through available hydroxyl groups and both DNA and RNA may be selectively acylated using similar techniques. The derivatizing agent may include a number of hydrophobic acyl groups.

Hydrophilic therapeutic and bioactive molecules may also be physically associated with the surface of a lipid particle through ionic interactions. An lipid particle with a net positive surface charge may be made using amphipathic surfactants comprising positively charged fatty acid chains. Such positively charged lipid particles will readily adsorb nucleic acids and other negatively charged compounds to the surface of the particle. Lipid particles with surface adsorbed DNA may be used for gene transfection vehicles in vitro and gene transfer agents for treating genetic diseases and for genetic vaccination.

A more preferred embodiment of the present invention includes a lipid particle comprising one or more hydrophobic solvents, one or more surfactants, one or more biologically active hydrophobic molecules with anti-cancer activity, and a polymeric shell, said polymeric shell comprised of one or more polymeric stabilizers. Lipid particles of this more preferred embodiment are useful for the administration of lipophilic anti-cancer agents. Simultaneous delivery of combinations of anti-cancer agents increases the benefit over monotherapies, and thus more effectively treats tumors. Furthermore, the combination of multiple hydrophobic anti-cancer agents may lead to synergistic anti-cancer activities.

One of the preferred anti-cancer agents useful in the lipid particle of the present invention is etoposide. Another of the preferred anti-cancer agents useful in the emulsions of the present invention is doxorubicin and its lipophillic derivatives thereof. Other preferred anti-cancer agents include daunorubicin, irenotecan, mitomycin, bleomycin, procarbazine, altretamine, and lipophilic pro-drug derivatives of methotrexate, hydrophobic cis-platin derivatives such as 2-hydrazino-4,5-dihydro-1H-imidazole with platinum chloride or 5-hydrazino-3,4-dihydro-2H-pyrrole with platinum chloride, vincristine, vinblastine, teniposide, epirubicin, camptothecin, teniposide, topotecan, etoposide, teniposide, monophosphoryl Lipid A, and muramyl dipeptide derivatives. Still other preferred anti-cancer agents useful in the practice of the present invention are taxanes, including but not limited to lipid-soluble taxane and taxane derivatives including paclitaxel; docetaxel; spicatin; taxane-2,13-dione, 5β-, 9β-, 10β-trihydroxy-, cyclic 9,10-acetal; taxane-2,13-dione, 5β-, 9β-, 10β-trihydroxy-,cyclic 9,10-acetal; taxane-2β-, 5β-, 9β-, 10β-tetrol, cyclic 9, 10-acetal; cephalomannine-7-xyloside; 7-epi-10-deacetylcephalomannine; 10-deacetylcephalomannine; cephalomannine; taxol B; 13-(2′,3′-dihydroxy-3′-phenylpropionyl) baccatin III; yunnanxol; 7-(4-Azidobenzoyl)baccatin III; N-debenzoyltaxol A; O-acetylbaccatin IV; 7-(triethylsilyl)baccatin III; 7,10-Di-O-[(2,2,2-trichloroethoxy)carbonyl]baccatin III; baccatin III 13-O-acetate; baccatin diacetate; baccatin; baccatin VII; baccatin VI; baccatin IV; 7-epi-baccatin III; baccatin V; baccatin I; baccatin III; baccatin A; 10-deacetyl-7-epitaxol; epitaxol; 10-deacetyltaxol C; 10-deacetyl-7-xylotaxol; 7-epi-10-deacetyltaxol; 10-deacetyltaxol; taxagafme and 10-deacetyltaxol B.

In a preferred embodiment of this invention, paclitaxel is present in an amount of about 0.1% to about 20% by weight of the lipid particle.

Another embodiment of the present invention is a lipid suspension comprising an aqueous solvent and a lipid particle, said lipid particle comprising one or more hydrophobic solvents, one or more surfactants, one or more biologically active hydrophobic molecules, and a polymeric shell, said polymeric shell comprised of one or more polymeric stabilizers. A preferred hydrophobic solvent is an oil. The lipid suspension of the present invention contain a stable discrete lipid particle entity in a hydrophilic milieu such as water, where the lipid particle protectively encapsulates one or more solubilized hydrophobic agents.

The aqueous solvent may further comprise drugs or pharmaceuticals which are soluble in aqueous solution. Combinations of drugs or pharmaceuticals in the hydrophobic phase and the aqueous phase may be formulated to yield a desired pharmaceutical composition.

In addition to the above constituents, the lipid suspension of this invention may include other pharmaceutically acceptable compounds or excipients to increase the stability of the lipid particles in suspension systems. The lipid suspension of this invention may include a suspending agent to disperse lipid particle evenly in an aqeuous milieu and to prevent the solid lipid particles from separating from the aqueous phase. Such pharmaceutically acceptable compounds or excipients include but are not limited to Xanthan Gum, tragacanth, cetyl alcohol, stearic acid, and/or beeswax (Remington's Pharmaceutical Sciences, 1975). Other preferred suspending agents include carboxymethylcellulose, methylcellulose, microcrystalline cellulose, poly(vinylpyrrolidone), and bentonite. In addition, tonicity and buffering agents to control pH of the suspending solution, as well as flavoring and coloring agents may be added.

Another embodiment of the present invention is a vaccine lipid suspension comprising an aqueous solvent and a lipid suspension, said lipid suspension comprising one or more hydrophobic solvents, one or more surfactants, one or more hydrophobic antigens, and a polymeric shell, said polymeric shell comprised of one or more polymeric stabilizers. A preferred hydrophobic solvent is an oil.

The vaccine lipid suspension of the present invention preferably further comprises one or more pharmaceutically acceptable excipients which are compatible with the said antigen. Suitable excipients include but are note limited to water, saline, dextrose, glycerol, ethanol, or combinations thereof.

The vaccine lipid suspension of the present invention preferably further comprises auxiliary substances including but not limited to wetting or emulsifying agents, and pH buffering agents.

The antigen of the present invention may be formulated into the vaccine lipid suspension as a neutral or salt form. Pharmaceutically acceptable salts include but are not limited to the acid addition salts (formed with free amino groups), which are formed with inorganic acids, including but not limited to hydrochloric or phosphoric acids, or organic acids such as acetic, oxalic, tartaric and maleic. Salts formed with free carboxyl groups are preferably derived from inorganic bases, including but note limited to sodium, potassium, ammonium, calcium, or ferric hydroxides, and such organic bases as isopropylamine, trimethylamine, 2-ethylamino ethanol, histidine, and procaine.

The vaccine lipid suspensions of the present invention may be multivalent or univalent. Many methods may be used to introduce the vaccine formulations of the present invention; including but not limited to oral, intradermal, intramuscular, intraperitoneal, intravenous, subcutaneous, intranasal, rectal, and via scarification (scratching through the top layers of skin, e.g., using a bifurcated needle). The patient to which the vaccine is administered is preferably an animal, more preferably a mammal, most preferably a human, but can also be a non-human animal including but not limited to cows, horses, sheep, pigs, fowl (e.g., chickens), goats, cats, dogs, hamsters, mice and rats.

The vaccine lipid suspensions of the present invention comprise an effective immunizing amount of one or more antigens and a pharmaceutically acceptable carrier or excipient. Pharmaceutically acceptable carriers are well known in the art and include but are not limited to saline, buffered saline, dextrose, Water, glycerol, sterile isotonic aqueous buffer, and combinations thereof. A further example of physiologically acceptable carrier is a physiologically balanced salt solution containing one or more stabilizing agents including but not limited to stabilized, hydrolyzed proteins and lactose. The pharmaceutically acceptable carrier is preferably sterile.

The vaccine lipid suspensions of the present invention may be in the form of a liquid solution, suspension, emulsion, sustained release formulation, powder, and preferably solid forms such as capsules, tablets or pills. Vaccine lipid suspensions for oral administration preferably include standard carriers including but not limited to pharmaceutical grades of mannitol, lactose, starch, magnesium stearate, sodium saccharine, cellulose, or magnesium carbonate.

Vaccine lipid suspensions in liquid form may be provided in a hermetically sealed container such as an ampoule or a sachet. The vaccine formulations are generally stored at 4° C. prior to use. The precise dose of vaccine lipid suspension to be employed will depend on the route of administration, and the nature of the patient, and should be decided according to the judgment of the practitioner and each patient's circumstances according to standard clinical techniques.

An effective dose of the immunogenic antigen is that amount sufficient to produce an immune response to the antigen in the host to which the vaccine lipid suspension is administered. Use of purified antigens as components of vaccine lipid suspensions may be carried out by standard methods. If the immunogenic antigen is a protein, the purified protein(s) should be adjusted to an appropriate concentration, formulated with any suitable vaccine adjuvant and encapsulated within the lipid suspension. Suitable adjuvants include but are not limited to mineral gels, such as aluminum hydroxide, surface active substances such as lysolecithin or pluronic polyols, polyanions, peptides, oil emulsions, alum, Lipid A and derivatives of Lipid A, cytokines, and MDP.

Another embodiment of the present invention includes methods for treating disease using a lipid suspension drug delivery system comprising administering to a mammal a lipid suspension in a suitable oral dosage form, such as a soft or hard-filled gelatin capsule, wherein said lipid suspension comprises an aqueous solvent and a lipid particle, said lipid particle comprising one or more hydrophobic solvents, one or more surfactants, one or more biologically active hydrophobic molecules, and a polymeric shell, said polymeric shell comprised of one or more polymeric stabilizers.

A more preferred embodiment of the present invention includes methods for treating cancer using a lipid suspension drug delivery system comprising administering to a mammal a lipid suspension in a suitable oral dosage form, such as a soft or hard gelatin capsule, wherein said lipid suspension comprises an aqueous solvent and a lipid particle, said lipid particle comprising one or more hydrophobic solvents, one or more surfactants, one or more biologically active hydrophobic molecules with anti-cancer activity, and a polymeric shell, said polymeric shell comprised of one or more polymeric stabilizers. The preferred anti-cancer agents are described above.

Another embodiment of the present invention includes methods for diagnosing a disease using a lipid suspension drug delivery system comprising administering to a mammal a lipid suspension in a suitable oral dosage form, such as a soft or hard gelatin capsule, wherein said lipid suspension comprises an aqueous solvent and a lipid particle, said lipid particle comprising one or more hydrophobic solvents, one or more surfactants, one or more biologically active hydrophobic molecules, and a polymeric shell, said polymeric shell comprised of one or more polymeric stabilizers.

The lipid particles and lipid suspensions of the present invention are preferably administered through mucosal tissue or epithelia The lipid particles and lipid suspensions of the present invention are therefore administered by those routes which optimize uptake by mucosa, such as sublingual, buccal, rectal and intranasal, and preferably oral. The lipid particles and lipid suspensions of the present invention can be delivered orally in the form of tablets, capsules, cachets, gelcaps, solutions, suspensions, topically in the form of creams, ointments, suppositories and the like, transdermally and parenterally.

If administered topically the lipid particles and lipid suspensions are preferably administered in the form of an ointment or transdermal patch. If administered intranasally the lipid particles and lipid suspensions are preferably administered in an aerosol form, spray, mist or in the form of drops. Suitable formulations can be found in Remington's Pharmaceutical Sciences, 16th and 18th Eds., Mack Publishing, Easton, Pa. (1980 and 1990), and Introduction to Pharmaceutical Dosage Forms, 4th Edition, Lea & Febiger, Philadelphia (1985), each of which is incorporated herein by reference.

The lipid particles and lipid suspensions of the present invention are suitable for administration to animals, preferably mammals and birds, and more preferably humans. For example, domestic animals such as dogs and cats, as well as domesticated herds, cattle, sheep, pigs and other domesticated mammals may be treated or vaccinated with the lipid particles and lipid suspensions of the present invention. In a preferred embodiment, the lipid particles and lipid suspensions of the present invention are administered to humans lipid particles and lipid suspensions are preferably provided in a hermetically sealed container such as an ampoule or sachet, and stored at 4° C. Dosages of the lipid particle and suspension compositions will vary depending on the individual patient and the mode of administration. Such dosages can be determined by a skilled physician using standard techniques.

The development of an oral formulation for an insoluble or poorly soluble drug often involves the designing of a system that will affect the pH of the micro-environment surrounding the drug form in the GI tract after ingestion. In particular, the formulation may contain disintegrants and/or other agents that work to increase or decrease the pH of the micro-environment, and thus enhance drug dissolution. In addition, the drug may also be granulated to reduce its particle size and/or increase the surface area that is exposed to the gastric fluid. The amount of exposed surface area will affect the rate of drug dissolution and thus the amount of active drug that will be absorbed by the patient. With respect to drug compounds of very poor or limited solubility, those skilled in the art have used co-solvents, surfactants or wetting agents to reduce the surface tension of the liquid environment of the gastric fluid in which the active drug is to be dissolved. These agents wet the active drug more quickly so that more of the drug is exposed to the gastric fluid in a shorter time, and may enhance its dissolution. Common types of surfactants and co-solvents that can be used include the cationic, anionic (e.g., sodium lauryl sulfate and gelatin), and nonionic types, as well as such co-solvents as the polyethylene glycols (PEGs). The role of the binder in the tablet drug form is to provide a tablet with sufficient hardness and integrity, but also must allow for sufficient disintegration and dissolution in the gastric environment. In this sense, a binder performs the opposite function of a disintegrant. The types of binders that can be used in drug formulations include gelatins of numerous grades, starches and starch derivatives (including corn starch, StaRx 1500, carboxymethylated starch), cellulose derivatives, polyvinylpyrollidones, Veegums, polyethylene glycols, sugars, e.g., sucrose and lactose, sodium alginate and waxes.

The fillers used to bulk up a drug tablet or other form also should not interfere with the tablet's dissolution. Numerous fillers include the starch derivatives, sugars (e.g., lactose and sucrose), sorbitol, mannitol, cellulose derivatives and their inorganic salts, corn starch, Starch 1500, calcium phosphate, and Avicel.

Likewise, lubricants aid in the machining of a drug tablet. Every tablet needs a lubricant so that it will be ejected from the machine die with minimum force. However, the lubricant also must not interfere with the dissolution of the tablet. Lubricants include waxes, fatty acids, sodium salts of fatty acids and stearates.

The invention is illustrated by the following non-limiting examples.

### EXAMPLE 1

### Polymerized And Non-Polymerized Lipid Suspensions Of Paclitaxel

6 milligrams of paclitaxel were added to 55 milligrams of polyethylene glycol 300 and 1.2 milligrams of poloxamer 188 and crystals were dissolved by mixing. At 45° C., 62 milligrams of soybean lecithin and soybean oil were mixed to which was added 3 milligrams of DODPC, which dissolved in the lecithin-soy oil. Paclitaxel-PEG was added in toto to the soy oil, forming a clear oil. In addition, 2 milligrams of HHP (2-hydroxy-4′-(2-hydroxyethoxy)-2-methylpropiophenone, Aldrich) were added as a polymerization initiator. The mixture was divided. One portion was left as an unpolymerized control. The other portion was exposed directly to ultraviolet light at 365 nanometers to polymerize the DODPC in the hydrophobic phase. Polymerization of DODPC was monitored by measuring the decrease of absorbance at 254 nanometers. The hydrophobic phase was polymerized until approximately 30% of the monomers had been reacted. The polymerized hydrophobic phase and the remaining portion not exposed to ultraviolet light were weighed and dispersed into approximately 2× the weight of the hydrophobic phase in water in which PEG 300 (10% w/v) had been dissolved and Xanthan Gum (0.3% w/v) to form lipid suspensions (formulations D, Table 2). The unpolymerized lipid spheres were divided into two portions: to one portion was added HHP and polymerized to 30% completion by exposure to ultraviolet light at 365 nanometers.

TABLE 1
Composition of Paclitaxel Lipid Particles

| | Lipid Particle Formulation (mg)¹ | | | |
|---|---|---|---|---|
| Component | D | E | F | G |
| Paclitaxel | 6 | 6 | 48 | 25 |
| Perillyl alcohol | — | 131 | 144 | 126 |
| Pluonric F68 (poloxamer 188) | 1.2 | 0.65 | 2.4 | 1.3 |
| Polyethylene glycol 300 | 188 | 185 | 168 | 75 |
| DODPC | 3 | 3 | 28 | 22 |
| Lecithin | 62 | 62 | 67 | 76 |
| Soy Oil | 62 | 62 | 68 | 73 |
| Xanthan Gum | 3 | 3 | 2.4 | 3.2 |
| Water | 653 | 617 | 490 | 631 |

¹Formulations D-G were both polymerized and unpolymerized

### EXAMPLE 2

### Lipid Particles With Perillyl Alcohol As A Solvent For Paclitaxel

Formulations E, F, and G (Table 2) were made similarly to formulations D with the exception that perillyl alcohol and PEG 300 was used to solubilize paclitaxel. Polymerization Formulations E-G were prepared as described for Formulation D. More paclitaxel was fully solubilized in the perillyl alcohol formulations than ones without.

### EXAMPLE 3

### Stability Of Paclitaxel Lipid Suspensions

Particle size was evaluated for Formulation D, E, F and G. Uniform distribution of particles with an average radius of approximately 100 nanometers was observed upon particle formation. Particle size stability was maintained at 4° C., at room temperature, and at 42°. for all particle formulations. Paclitaxel crystals were not observed microscopically in any of the formulations.

### EXAMPLE 4

### Bioavailability Of Paclitaxel Following Intraduodenal Administration Of Lipid Particles In Rats

Sprague-Dawley rats (approximately weighing 220 grams each) were catheterized surgically with jugular and duodenal catheters. Each group of rats, 3 animals per group, were given 6 mg/kg of paclitaxel in formulation D. Blood samples were collected at 0, 20, 40, 60, 90, 120, and 240 minutes following administration of the formulations. The time 0 blood collection was obtaintd approximately 15 minutes before experimental application of formulations. Plasma samples were analyzed by a solid phase extraction of paclitaxel followed by HPLC. Pharmacokinetic parameters were calculated from the data using WinNonLin software (Pharsight). Approximately 100 ng/ml peak plasma concentrations of paclitaxel was observed in all rats.

### EXAMPLE 5

### Tumor Regression By Oral Administration Of Paclitaxel As A Lipid Suspension

Athymic nude mice are injected subcutaneously with approximately 10⁷ MDA-MB-231 cells. Tumors develop at the injection site until they are approximately 100 mm³ in size. Mice are treated by intraperitoneal injection of cremophor paclitaxel (commercial formulations from Bristol Myers Squibb consisiting of ethoxylated Castor oil and ethanol to dissolve paclitaxel) or cremophor alone as controls. Subject mice are given doses of paclitaxel as lipid suspensions by oral gavage once a day. Tumor size is measured and proportion of mice with tumor regression is measured.

### EXAMPLE 6

### Effect Of Lipid Suspensions On Human Breast Cancer Cell Lines

Human breast cancer cell lines are implanted subcutaneously into nude mice. Three human cell lines, MCF-7, BT-20, and MDA-MB-231 are used. Tumors are harvested and cells are grown in RPMI supplemented with fetal bovine serum (10%), ampicillin (100 micrograms per ml), streptomycin, (100 micrograms per ml), and glutamine (0.3%). The cells are grown to approximately 80% confluence and treated with paclitaxel in Cremophor, Cremophor solution alone, dilution of lipid suspension formulations without paclitaxel, or paclitaxel in lipid suspension formulations. Viable cells are determined at times after addition by enumerating proportion of living cells by dye exclusion technique using tetrazolium blue.

What is claimed is:

1. A particle composition that comprises
(a) a hydrophobic phase;
(b) a surfactant;
(c) a biologically active agent; and
(d) a polymeric stabilizer.

2. The particle composition of claim 1 wherein the hydrophobic phase comprises an oil or mixture of oils.

3. The particle composition of claim 2 wherein the oil is a triglyceride.

4. The particle composition of claim 1 wherein the hydrophobic phase comprises a triglyceride that is liquid between 10° C. and 45° C.

5. The particle composition of claim 1 wherein the hydrophobic phase comprises a triglyceride that is liquid between 10° C. and 70° C.

6. The particle composition of claim 1 wherein the surfactant is selected from the group consisting of an alkyl glycerolphosphoryl choline, a polyoxyethylene polymer, a block copolymer of polyoxyethylene and polyoxypropylene, and an ethoxylated glycerol ester.

7. The particle composition of claim 1, wherein the surfactant comprises one or more glycerol fatty acid esters or hydrophilic derivatives thereof.

8. The particle composition of claim 7, wherein the fatty acid esters have a length of about 6 to about 12 carbon atoms.

9. The particle composition of claim 1, wherein surfactant comprises a monoglyceride, diglyceride or mixture thereof.

10. The particle composition of claim 1, wherein the surfactant comprises a monoglyceride or a hydrophilic derivative or analog thereof.

11. The particle composition of claim 1, wherein the surfactant comprises a diglyceride or a hydrophilic derivative or analog thereof.

12. The particle composition of claim 1, wherein the surfactant comprises a monoglyceride or diglyceride mixture.

13. The particle composition of claim 6, wherein the surfactant further comprises a diglyceride or a hydrophilic derivative or analog thereof.

14. The particle composition of claim 1, wherein the particle suspension further comprises a solvent selected from the group consisting of water, glycerol, sorbitol, mannitol, propylene glycol, ethylene glycol, polyethylene glycol or mixtures thereof.

15. The particle composition of claim 1, wherein the particle suspension further comprises a solvent selected from the group consisting of a monoterpene or a derivative thereof.

16. The particle composition of claim 15, wherein the monoterpene is perillyl alcohol, perillic acid or d-limonene.

17. The particle composition of claim 10, wherein the monoglyceride is derivatized by a polyoxyethylene polymer.

18. The particle composition of claim 10, wherein the monoglyceride is derivatized by a polyoxyethylene polymer from about 200 to about 10,000 in molecular weight.

19. The particle composition of claim 10, wherein the monoglyceride is derivatized by a polyoxyethylene polymer from about 200 to about 4,000 in molecular weight.

20. The particle composition of claim 11, wherein the diglyceride is derivatized by a polyoxyethylene polymer.

21. The particle composition of claim 3, wherein at least one of the fatty acid esters of said triglyceride is derivatized with acetic acid, citric acid, lactic acid, succinic acid, tartaric acid or mixtures thereof.

22. The particle composition of claim 7, wherein at least one of the fatty acid esters is a caprylic acid or capric acid.

23. The particle composition of claim 1, wherein the polymeric stabilizer is a natural polymer, a synthetic polymer or a mixture thereof.

24. The particle composition of claims 1 or 23, wherein the polymeric stabilizer is a polymerizable fatty acid or phospholipid.

25. The particle composition of claim 24, wherein the polymeric stabilizer is ODP or OPS.

26. The particle composition of claim 24, wherein the polymer is formed from a polymerizable fatty acid monomer.

27. The particle composition of claim 1 wherein said composition is in the form of a polymer, said polymer formed by interfacial ionic polymerization with water or other initiators.

28. The particle composition of claim 23, wherein said composition is in the form of a polymer, said polymer formed by condensation of cyanoacrylates, including alkylcyanoacrylates.

29. The particle composition of claim 23, wherein the polymer is formed from condensation of ethyl 2-cyanoacrylate.

30. The particle composition of claim 23, wherein the synthetic polymer is selected from the group consisting of polylactide, polyglycolide, a mixture of polylactide and polyglycolide, a polycaprolactone,a polyortho esters, polysebacic acid, polyfumaric acid, polyamides, polycarbonates, polyalkylenes, polyacrylamides, poly(hydroxy acids), polyanhydrides, polyorth oesters, polyacrylate, polyvinyl alcohols, blends or copolymers thereof.

31. The particle composition of claim 1, wherein the particle is suspended in a aqueous phase comprising an additive selected from:the group consisting of a suspending agent, buffering agent, a tonicity agent, an oxidizing agent, a reducing agent, an antimicrobial agent, a preservative, a stabilizing agent, or a mixture thereof.

32. The particle composition of claim 1, wherein the hydrophobic phase is present in an amount from about 0% to about 50% by weight of the particle composition.

33. The particle composition of claim 1, wherein the hydrophobic phase is present in an amount from about 0% to about 50% by weight of the particle composition.

34. The particle composition of claim 1, wherein.the hydrophilic phase is present in an amount from about 10% to 25% by weight of the particle composition.

35. The particle composition of claim 1, wherein the surfactant has an HLB value of about 1 to about 45.

36. The particle composition of claim 1, wherein the surfactant has an HLB of about 1 to about 20.

37. The particle composition of claim 1, wherein the biologically active molecule is a prophylactic or therapeutic agent.

38. The particle composition of claim 1, wherein the biologically active molecule is a diagnostic agent.

39. The particle composition of claim 37, wherein the therapeutic agent is a taxane or an analog thereof.

40. The particle composition of claim 1 or 37, wherein the therapeutic agent is paclitaxel.

41. The particle composition of claim 1, wherein the biologically active molecule is a topisomerase inhibitor.

42. The particle composition of claim 1, wherein the biologically active molecule is more than about 0.1 mg/ml soluble in the hydrophobic phase.

43. The particle composition of claim 1, wherein the biologically active molecule is more than about 1 mg/ml soluble in the hydrophobic phase.

44. The particle composition of claim 1, wherein said composition is in the form of a suspension.

45. The particle composition of claim 44, wherein the particle size of said suspension is in the range of about 10 nm to 10,000 about nm.

46. The particle composition of claim 44, wherein the particle size of said suspension is in the range of about 10 nm to about 1,000 nm.

47. A method for preventing, treating or ameliorating one or more symptoms associated with a disease or disorder in an animal comprising administering to an animal a composition according to any of claims 1-23, 25-39, 41-44, or 45.

48. A method for preventing, treating or ameliorating one or more symptoms associated with a disease or disorder in an animal comprising administering to an animal a composition according to claim 24.

49. A method for preventing, treating or ameliorating one or more symptoms associated with a disease or disorder in an animal comprising administering to an animal a composition according to claim 40.

50. A method for diagnosing a disease or disorder in an animal comprising administering to said animal a composition according to any one of claims 1-23, 25-39, 41-44, or 45.

51. A method for diagnosing a disease or disorder in an animal comprising administering to said animal a composition according to claim 24.

52. A method for diagnosing a disease or disorder in an animal comprising administering to said animal a composition according to claim 40.

53. The method according to any one of claims 47-50, or 51 wherein the composition is administered as a capsule, soft elastic gelatin capsule, caplet, aerosol, spray, solution, suspension, emulsion, sachet, tablet, capsule, powder or granules.

54. The method according to any one of claims 47-52, or 53, wherein said mammal is a human.

55. The method according to any one of claims 47-52, or 53, wherein the composition is administered orally.

56. The method according to any one of claims 47-52, or 53, wherein the polymeric stabilizer is a natural polymer, a synthetic polymer or a mixture thereof.

57. The particle composition of claim 41 wherein the topoisomerase inhibitor is selected from the group consisting of etoposide, camptothecin, topotecan or a derivative thereof.

58. A method for orally administering paclitaxel to a patient comprising administering the composition of claim 40 to a patient at a dose of paclitaxel in the range of about 10 mg/m² to about 1,000 mg/m².
